(19) United States
(12) Patent Application Publication
(10) Pub. No.: US 2021/0102151 A1
(43) Pub. Date: Apr. 8, 2021

(54) BIONIC ORGAN DEVICE AND METHOD FOR MAKING THE SAME

(71) Applicant: Chung Yuan Christian University, Taoyuan City (TW)

(72) Inventors: Tzong-Rong Ger, Taoyuan City (TW); Chun-Kai Huang, Taoyuan City (TW); Wu-Zhang Su, Taoyuan City (TW); Tan-Yueh Chen, Taoyuan City (TW); Ji-Ling Zeng, Taoyuan City (TW); Nathalia Mutiara, Muara Enim (ID)

(21) Appl. No.: 17/031,852

(22) Filed: Sep. 24, 2020

(30) Foreign Application Priority Data
Oct. 2, 2019 (TW) 108135673

(51) Int. Cl. C12M 1/12; C12M 1/00; C12N 5/071; C12M 1/34

(52) U.S. Cl. CPC C12M 25/02; C12M 23/20; C12M 41/12; C12N 5/068; C12M 25/14

(57) ABSTRACT

The present invention provides a bionic organ device comprises a porous thermo-responsive layer, a cell culturing layer, a flow channel and a controlling module. The thermo-responsive layer is formed by weaving a fiber made of/from a plurality of thermo-responsive polymers and has a first surface and a second surface opposite to the first surface. The cell culturing layer is formed on the first surface of the thermo-responsive layer. The flow channel has an accommodating space for accommodating the thermo-responsive layer, wherein the flow channel is utilized to allow a flow passing through the second surface inside the flow channel. The controlling module is utilized to allow the flows having different flow temperatures passing through the second surface in the flow channel so as to control a temperature variation of the thermo-responsive layer around critical temperature whereby an expansion and contrast motion of the thermo-responsive layer can be generated.

This application claims the benefit of Taiwan Patent Application Serial No. 108135673, filed Oct. 2, 2019, the subject matter of which is incorporated herein by reference.

### BACKGROUND OF INVENTION

### 1. Field of the Invention

The present invention is related to a bionic technique, and, more particularly, to a bionic organ device for simulating a contraction movement or an expansion movement of organ, and a method for making the bionic device.

### 2. Description of the Prior Art

Before reaching the market, it takes much cost and time for the drug candidates to pass successfully through drug screening and clinic trials. In the clinic trial process, the animal experimentation is inevitable. Since the Animal Protection Act is executed in the recent years, the execution of animal experimentation is getting stricter. In addition, not only the cost is getting higher, but also the dispute could be induced. Accordingly, there is a need to generate a totally new inspection process for replacing the animal experimentation and reducing the cost.

In the prior arts, such as “Reconstituting Organ-Level Lung Function on a Chip.”, Science. DOI: 10.1126/science.1188302, provided by Dongeun (Dan) Hub, or “Human Breathing Lung-on-a-Chip” American Thoracic Society, 2015 March; 12(Suppl 1): S42-S44, for example, those arts disclosed a cell development technology for cultivating cells in a simulated human organ environment. It is found that the characteristic of the cells cultured by the simulation environment are more close to the real organ cells of human being.

By using the above-mentioned technology, a bionic device for simulating the interaction between the alveoli and micro-capillaries can be manufactured. For example, please refer to FIGS. 1A and 1B, the epidermal cells 90 and endothelial cells of the alveoli can be cultured on a porous thin film 92 that is stretchable whereby the interaction between alveoli and micro-capillaries during breath could be reproduced. The thin film 92 is coupled to the channel 93 having vacuum effect exerting thereon so that a stretch and contraction of alveoli could be simulated through the thin film 92. Although the technologies of the above-mentioned prior arts are capable of producing bionic organ, the manufacturing process of control valve is complicated and difficult.

Moreover, the U.S. Pat. No. 9,725,687 and US.Pub.NO. US20140342445 are also disclosed the related technology for creating bionic device performing the similar application.

### SUMMARY OF THE INVENTION

The present invention provides a bionic organ device and method for making the same in which the device is formed by the thermo-responsive layer so that when the temperature of the thermo-responsive layer is controlled to be varied around the critical temperature of the thermo-responsive layer, e.g. above or below the critical temperature, the thermo-responsive layer could be contracted or expanded due to the variation of temperature so that the thermo-responsive layer can be utilized to simulate the movement of organ for physiology research or drug development.

The present invention provides a bionic organ device and method for making the same, in which the porous thermo-responsive layer could be formed by electro-spinning process. The spinning could be utilized to weave the film material having hydrophilic and hydrophobic characteristics for performing contraction or expansion due to the temperature variation. Alternatively, the porous thermo-responsive layer could also be formed by thermo-responsive polymer molecules and UV curable glue.

In one embodiment, the present invention provides a bionic organ device comprises a porous thermo-responsive layer, a cell culturing layer, a flow channel and a flow control module. The porous thermo-responsive layer is configured to have a porous structure formed by a plurality of thermo-responsive polymer molecules. In addition, the porous thermo-responsive layer comprises a first surface and a second surface opposite to the first surface. The cell culturing layer is formed on the first surface of the porous thermo-responsive layer. The flow channel is configured to have an accommodating space for accommodating the porous thermo-responsive layer, and to allow at least one flow passing through the second surface of the porous thermo-responsive layer. The flow control module is configured to control the flow having different temperature passing through the second surface of the porous thermo-responsive layer such that a temperature of the porous thermo-responsive layer is varied between an expansion temperature and a contraction temperature of the porous thermo-responsive polymer molecules thereby causing an expansion movement and contraction movement generated by the porous thermo-responsive layer for simulating an organ effect.

In one embodiment, a method for forming a bionic organic device, comprising steps of forming a porous thermo-responsive layer having a plurality of thermo-responsive polymer molecules, the porous thermo-responsive layer comprising a first surface and a second surface opposite to the first surface, forming a cell culturing layer on the first surface of the porous thermo-responsive layer such that the cell culturing layer and the porous thermo-responsive layer are formed as a bionic structure layer, arranging the bionic structure layer into a flow channel having an accommodating space for accommodating the porous thermo-responsive layer, wherein the flow channel is configured to allow at least one fluid passing through the second surface of the porous thermo-responsive layer and selecting the fluid having different temperature passing through the second surface of the porous thermo-responsive layer by using a flow control module coupled to the flow channel whereby the porous thermo-responsive layer generates a contraction or an expansion movement due to a temperature variation of the porous thermo-responsive layer over or under a critical temperature with respect to expansion movement and contraction movement of the porous thermo-responsive layer.

In one embodiment, the flow control module further comprises a first flow, a second flow, and a valve module wherein the first flow has a first temperature less than or equal to the expansion temperature or the contraction temperature of the thermo-responsive polymer molecules, the second flow has a second temperature greater than or equal to the expansion temperature or the contraction temperature of the thermo-responsive polymer molecules, and the valve module is coupled to the flow channel for selectively communicate the first flow or the second flow to the flow channel thereby changing the temperature of the porous thermo-responsive layer.

In an alternative embodiment, a cell culturing fluid is arranged on the top of the cell culturing layer in the flow channel whereby the cell culturing layer absorbs a plurality of cells.

In an alternative embodiment, the step for forming the porous thermo-responsive layer further comprises steps of forming a spinning by using the thermo-responsive polymer molecules and weaving the spinning to form the porous thermo-responsive layer, wherein the step of weaving the spinning is a vertical weaving, a horizontal weaving or a random weaving.

### BRIEF DESCRIPTION OF THE DRAWINGS

The present invention will now be specified with reference to its preferred embodiment illustrated in the drawings, in which:

FIGS. 1A and 1B illustrate a conventional bionic device for controlling the contraction and expansion;

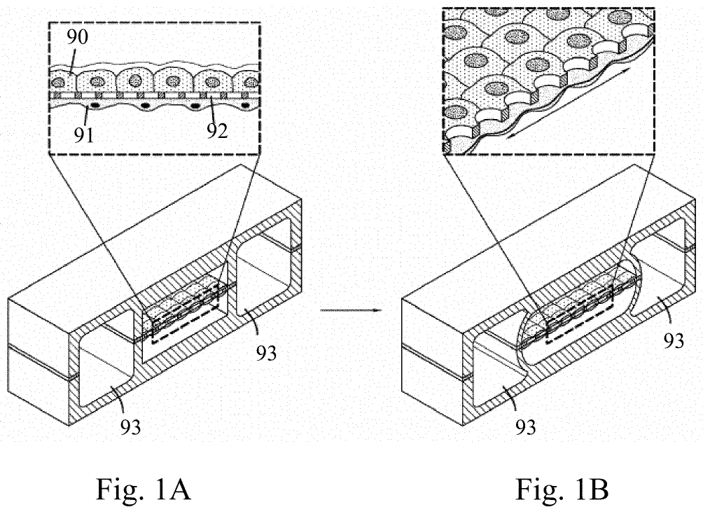

FIG. 2A illustrates a bionic organ device according to one embodiment of the present invention;

FIG. 2B illustrates a combination of a flow channel and porous thermo-responsive layer of the bionic organ device according to one embodiment of the present invention;

FIGS. 2C-2D respectively illustrate alternative bionic organ devices according to another embodiment of the present invention;

FIGS. 3A to 3C illustrate weaving process for forming the porous thermo-responsive layer according to different kinds of embodiments of the present invention;

FIGS. 3D to 3E illustrate porous thermo-responsive layer according to different embodiments of the present invention;

FIGS. 4 and 5 illustrate valve module according to different embodiments of the present invention;

FIGS. 6 and 7 illustrate contraction and expansion movement of the porous thermo-responsive layer of the present invention;

FIG. 8 illustrates a method for forming bionic organ device according to one embodiment of the present invention;

FIG. 9A illustrates a curve representing the variation of contraction rate according to one embodiment of the porous thermo-responsive layer formed by random spinning weaving;

FIG. 9B illustrates a curve representing the variation of contraction rate according to one embodiment of the thermo-responsive layer formed by horizontal spinning weaving;

FIG. 9C illustrates a curve representing the variation of contraction rate according to one embodiment of the thermo-responsive layer formed by vertical spinning weaving; and

FIGS. 10A to 10C illustrate a curve representing a variation of contraction and expansion during the operation time of first flow or the second flow.

### DESCRIPTION OF THE PREFERRED EMBODIMENT

The invention disclosed herein is directed to bionic organ device and method for making the same. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

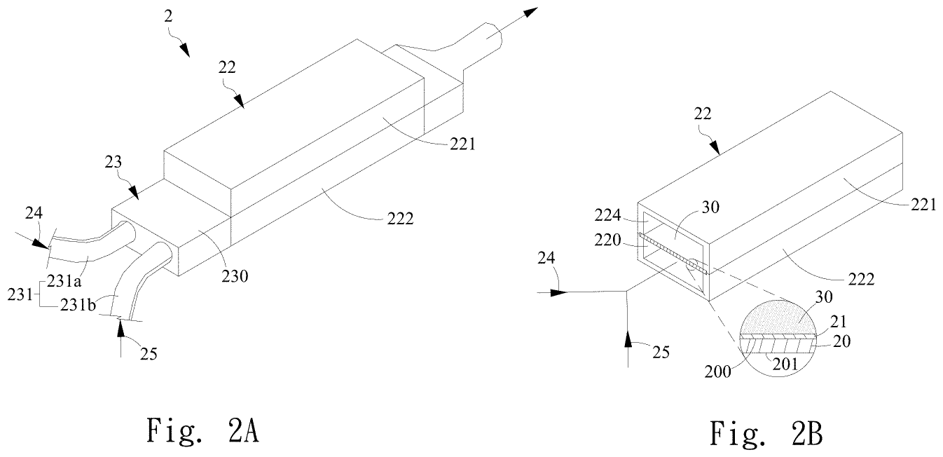

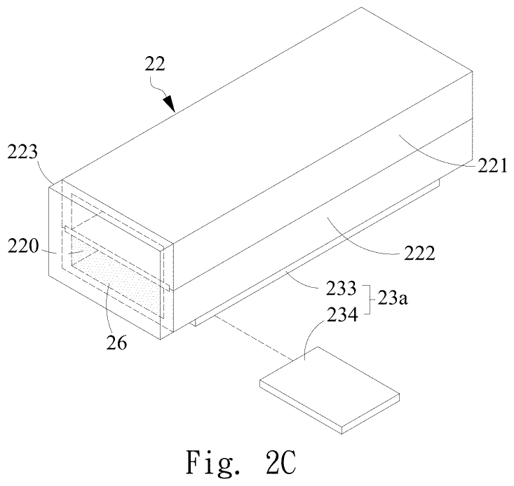

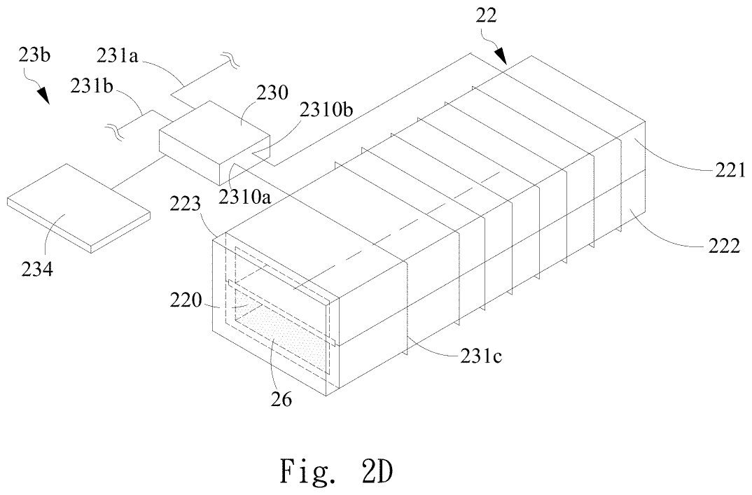

Please refer to FIGS. 2A and 2B, wherein the FIG. 2A illustrates a bionic organ device according to one embodiment of the present invention, and FIG. 2B illustrates a combination of flow channel and porous thermo-responsive layer of the bionic organ device. The bionic organ device 2 comprises a porous thermo-responsive layer 20, a cell culturing layer 21, a flow channel 22, and a control module 23. In the present embodiment, the porous thermo-responsive layer 20 is formed by weaving spinning formed by a plurality of thermo-responsive polymer molecules.

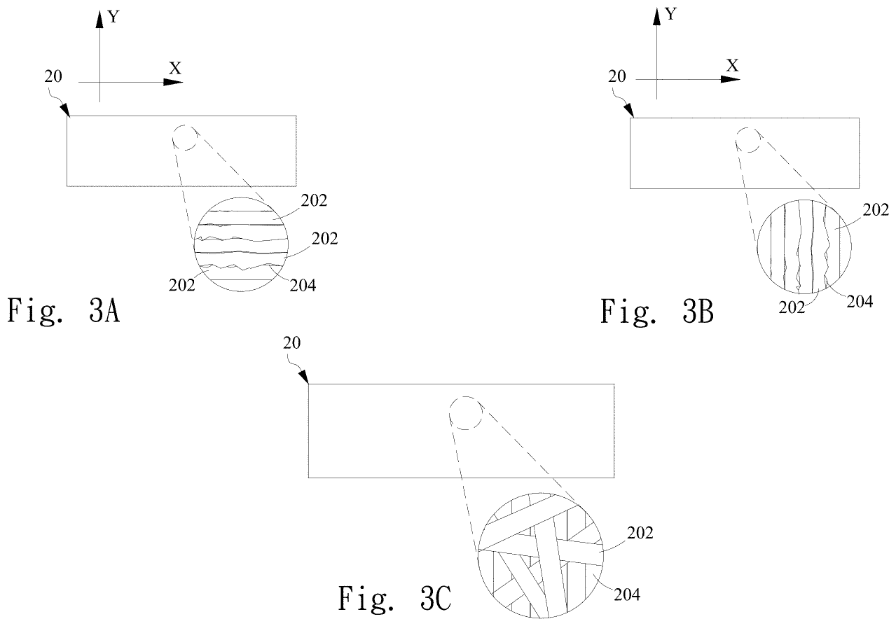

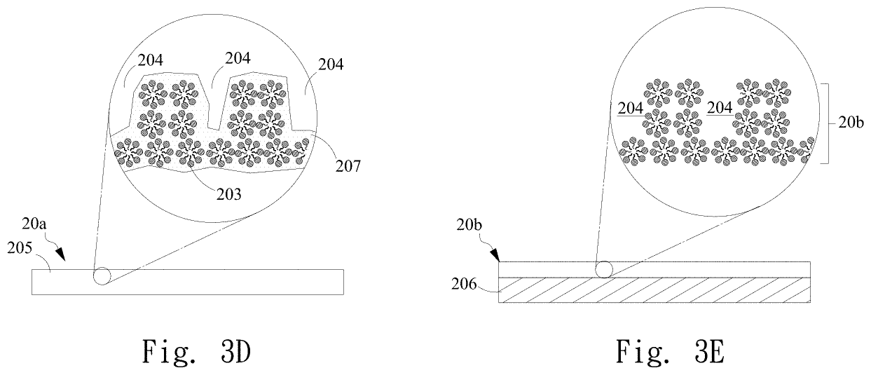

The porous thermo-responsive layer 20 has a first surface 200 and a second surface 201 opposite to the first surface 200. In one embodiment, the porous thermo-responsive layer 20 could be formed by horizontally weaving the spinning. Please refer to the FIG. 3A, in this embodiment, each spinning 202 extends along X direction and arranged one by one along the Y direction. It is clear that a plurality of porous structure 204 are formed at the boundary between the two adjacent spinning 202, or formed at the intersected area between spinning 202. Alternatively, the porous thermo-responsive layer could be formed by vertically weaving the spinning. Please refer to the FIG. 3B, in this embodiment, each spinning 202 extends along Y direction and arranged one by one along the X direction. It is clear that a plurality of porous structure 204 are formed at the boundary between the two adjacent spinning, or formed at the intersected area between spinning. Moreover, in another embodiment, the porous thermo-responsive layer 20 could be formed by randomly weaving the spinning. Please refer to the FIG. 3C, in this embodiment, each spinning 202 randomly intersected with the other spinning without regular arrangement. It is clear that a plurality of porous structure 204 are formed at the boundary between the two adjacent spinning 202, or formed at the intersected area between spinning 202. In one embodiment, the thermo-responsive polymer molecules could be, but should not be limited to, poly(N-Isopropylacrylamide). Alternatively, other polymer molecules having hydrophilic and hydrophobic characteristics could also be selected as the material for forming the porous thermo-responsive layer according to the user's need.

Please refer to FIGS. 2A and 2B, the cell culturing layer 21 is formed on the first surface 200 of the porous thermo-responsive layer 20 such that the combination of the porous thermo-responsive layer 20 and the cell culturing layer 21 can be regarded as a bionic structural layer. The material for forming the cell culturing layer 21 could be, but should not be limited to, polyethylenimine (PEI). The material could be changed to another proper material according to the user's need. The flow channel 22 is configured to have an accommodating space 220 for accommodating the porous thermo-responsive layer 20 and the cell culturing layer 21.

In the present embodiment, the flow channel 22 is configured to allow at least one flow passing through the second surface 201 of the porous thermo-responsive layer 20. In one embodiment, the flow channel 22 comprises a top housing 221 and bottom housing 222 assembled with the top housing 221 for forming the flow channel 22. The bionic structural layer is arranged between the top and bottom housings 221 and 222. It is noted that the structure of the flow channel 22 is not limited to the embodiment shown in the FIGS. 2A and 2B. It is noted that the flow could be a liquid flow or gas flow.

The control module 23 is utilized to allow one certain type flow flowing into the accommodating space 220 and passing through the second surface 201 whereby the temperature of the porous thermo-responsive layer 20 could be changed to be above or below a critical temperature of the porous thermo-responsive layer 20 by the flow in the flow channel 22 whereby a contraction movement and an expansion movement of the porous thermo-responsive layer 20 can be generated. The critical temperature can be ranged between 32-34.4° C. In the present embodiment, the critical temperature is 32° C.

It is noted that the critical temperature is varied according to the property of material forming the porous thermo-responsive layer 20 so it is not limited to the above-mentioned range. In addition, the critical temperature can be lower critical solution temperature (LCST), upper critical solution temperature (UCST), or other temperature or temperature range that can enable the porous thermo-responsive layer 20 to generate contraction movement or expansion movement. In the present embodiment, the LCST of the porous thermo-responsive polymer layer is 32° C. It is noted that the value or range of LCST depends on the material property of temperature-responsive polymer and it is not limited to the exemplary temperature described hereto. When the temperature of the porous thermo-responsive layer is higher than LCST, for example 37° C., the porous thermo-responsive layer 20 will be converted into a hydrophobic status and become contracted status. On the contrary, when the temperature of the porous thermo-responsive layer is lower than LCST, such as 28° C., for example, the porous thermo-responsive polymers 20 will be converted into hydrophilic status and become expanded.

The control module 23 is configured to select flows respectively having different flow temperature and enable the selected flow to pass through the space corresponding to the second surface 201 of the porous thermo-responsive layer 20 whereby the porous thermo-responsive layer 20 can be contracted or expanded. It is noted that the flow could be directly contact with the second surface 201, or having an interface contacting with the second surface 201. In the present embodiment, the control module 23 comprises a valve module 230 coupled to the flow channel 22, and a pipe module 231 coupled to the vale module 230.

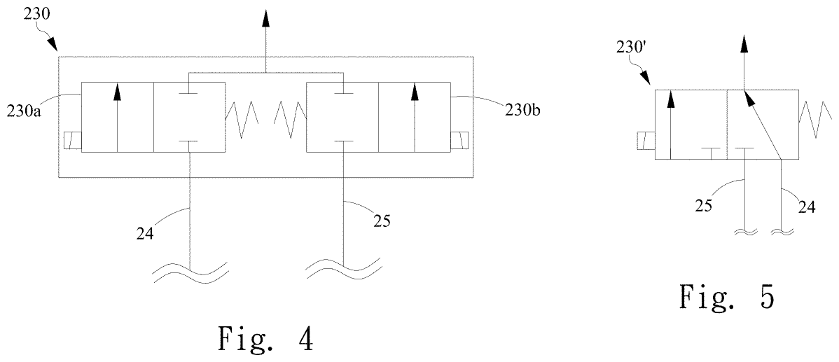

In the present embodiment, the pipe module 231 comprises a first pipe 231a and a second pipe 231b, wherein the first pipe 231a is configured to guide the first flow 24 flowing into the valve module 230, and the second pipe 231b is configured to guide the second flow 25 flowing into the valve module 230. Please refer to the FIG. 4, in one embodiment, the valve module 230 has two 2/2 way valves 230a, and 230b, in which the 2/2 way valve 230a is utilized to control the first flow 24, while the 2/2 way valve 23b is utilized to control the second flow 25. Alternatively, in another embodiment shown in FIG. 5, the valve module 230′ is a 3/2 way valve.

In the embodiment shown in FIG. 5, the first flow 24 is a predetermined flow flowing into the channel 22 while the second flow 25 is switched to flow into the channel 22 when there is a need to change the temperature of the porous thermo-responsive layer. It is noted that the design of the valve modules are not limited to the embodiments shown in FIG. 4 and FIG. 5. The user could select and arrange the appropriate valve elements according the operation need. In addition, the valves shown in FIGS. 4 and 5, can be, but should not be limited to, electromagnetic valves.

Please refer to FIGS. 2A and 2B, a cell culturing liquid 30 can be filled into the space 224 between the flow channel 22 and the cell culturing layer 21, whereby the upper side of the cell culturing layer 21 could adhere the plurality of cells from the cell culturing liquid 30. In the present embodiment, the first flow 24 has a first temperature smaller than the critical temperature with respect to expansion movement and contraction movement of the porous thermo-responsive layer 20 while the second flow 25 has a second temperature greater than the first temperature. The second temperature is greater or equal to the critical temperature with respect to the expansion movement and contraction movement of the porous thermo-responsive layer 20. Alternatively, the space 224 can also filled with medical liquid or other testing liquid for therapy or medical treatment.

In one embodiment, the valve module 230 is controlled to allow the first flow 24 or the second flow 25 flowing into the channel 22 such that the first flow 24 or the second flow 25 could have a heat transfer effect with the porous thermo-responsive layer 20 thereby increasing or decreasing the temperature of the porous thermo-responsive layer 20. When the temperature of the porous thermo-responsive layer 20 is above or below the critical temperature with respect to the expansion movement or contraction movement of the porous thermo-responsive layer, the porous thermo-responsive layer 20 is expanded or contracted due to the variation of temperature. In the present embodiment, the first flow 24 or the second flow 25 can be, but should not limited to, water.

It is noted that the method for forming the porous thermo-responsive layer 20 of the present invention is not limited to weaving the spinning. Alternatively, in another embodiment shown in FIG. 3D, the porous thermo-responsive layer 20a is formed by a self-assembly thermo-responsive layer 205 having a plurality of thermo-responsive polymer molecules 203 stacking with each other. In the present embodiment, the self-assembly means that porous thermo-responsive layer 205 is formed by enabling the temperature of the thermo-responsive polymer molecules greater than the critical temperature of the contraction movement such that the molecules are dehydrated to contracted thereby a space between the thermo-responsive polymer molecules can be generated for forming the porosity, coating an adhesive layer 207, such as ultraviolet (UV) curable glue or adhesive material, for example, onto the contracted thermo-responsive polymer molecules 203, and finally, the adhesive layer 207 is cured through UV light or dried naturally.

Alternatively, in another embodiment shown in FIG. 3E, a thermo-responsive layer 20b is formed on a substrate 206 or substrate having porosity through a titrating or spinning coating process. In another embodiment, a press mold having a plurality of micro structures, such as micro protrusion structures, for example, is utilized to press onto the thermo-responsive layer 20b, whereby a plurality of porous structures 204 is formed on the thermo-responsive layer 20b. The material for forming the press mold can be, but should not be limited to, ceramic material, metal material, or glass material. It is noted that the substrate 206 is non-essential element, which could be removed after forming the thermo-responsive layer 20b or forming the porous structures 204. In addition, the substrate 206 could also be the substrate having micro-structures protruded from the surface of the surface of the substrate. When the thermo-responsive polymer is coated or titrated on to the substrate, a plurality of porous structures could be formed on the thermo-responsive layer.

Alternatively, the flow could only be a single flow enclosed within the flow channel 22. Please see the FIG. 2C, which illustrates an alternative bionic organ device according to another embodiment of the present invention. In this embodiment, the flow or liquid 26 is arranged in the space 220 of channel 22 for contacting with the second surface of the porous thermo-responsive layer 20. A cover 223 is arranged to seal the opening of the channel 22 after the flow or liquid is filled into the space 220. The control module 23a in the present embodiment is an energy controlling module having heating and cooling effects acting on the flow inside the space 220 whereby the temperature of the flow in the channel 22 can be increased or decreased. With the design of the cover 223, the flow or liquid 26 could be replaced.

In the present embodiment, the energy controlling module has a thermoelectric cooling module 233 and a controller 234 electrically coupled to the thermoelectric cooling module 233. The thermoelectric cooling module 233 is arranged on the housing corresponding to the flow in the channel 22, whereby the thermoelectric cooling module 233 can be utilized to cool down the temperature of the flow in the channel 222 or to heat the flow thereby increasing the temperature of the flow. Accordingly, the temperature of the flow can be controlled to be above or below the critical temperature of the thermo-responsively the porous thermo-responsive layer 20 whereby an expansion movement and contraction movement is generated by the porous thermo-responsive layer. The controller 234 could be, but are not limited to, a controlling chip, a computer, smart phone, notebook, or devices that could control the thermoelectric cooling module 233 directly or through a user interface, such as graphical user interface, for example.

Alternatively, please refer to FIG. 2D, in the present embodiment, the different part from the embodiment shown in FIG. 2C is the control module. In the present embodiment, the control module 23b has a valve module 230 coupled to the flow channel 22, and a pipe module 231 coupled to the vale module 230. The pipe module 231 comprises a first pipe 231a and a second pipe 231b, wherein the first pipe 231a is configured to guide the first flow 24 having a first temperature flowing into the valve module 230, and the second pipe 231b is configured to guide the second flow 25 having a second temperature flowing into the valve module 230.

The pipe module 231 also has a third pipe 231c wrapping around the housing of the channel 22. The two ends 2310a and 2310b are communicated with the valve module 230 whereby the flow 24 or flow 25 entering the valve module 230 could be guided into the third pipe 231c from the end of 2310a and returns into the valve module 230 through the end 2310b. The flows 24 and 25 could be utilized to change the temperature of the flow 26 inside the channel. In addition, the controller 234 is electrically coupled to the valve module 230. The controller 234 utilized to control the valve module 230 could be, but are not limited to, a controlling chip, a computer, smart phone, notebook, or devices that could control the thermoelectric cooling module 233 directly or through a user interface, such as graphical user interface, for example.

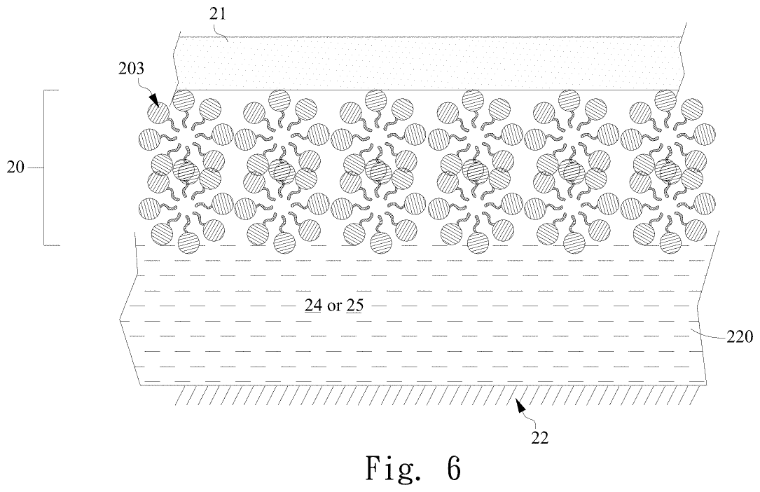

Next the operation principle of the present invention is explained below. Please refer to the FIGS. 2A, 6 and 7, which illustrate a contraction movement and an expansion movement according to one embodiment of the present invention. In FIG. 6, at a first time point, the first flow 24 is switched to flow into the space 220 between the flow channel 22 and the second surface 201 of the porous thermo-responsive layer 20. In the present embodiment, the temperature of the first flow 24 is smaller than the LCST, which is 5° C., for example. During the first flow 24 flow into the channel 22, the heat transfer effect is operated between the first flow 24 and the porous thermo-responsive layer such that the temperature of the porous thermo-responsive layer is below the LCST so that the molecules of the porous thermo-responsive layer absorbs the liquid whereby an expansion effect is occurred to stretch the whole porous thermo-responsive layer 20.

Please refer to FIG. 7, at a second time point, the valve module 230 is switched to select the second flow 25 so that the second flow 25 flows into the flow channel 22 for contacting with the second surface of the porous thermo-responsive layer 20 in the flow channel 22. Since the temperature of the second flow 25 is greater than LCST, e.g. 32° C., in the present embodiment, the temperature of the second flow 25 is 40° C. When the second flow 25 contacts with the porous thermo-responsive layer 20, the thermo-responsive polymer becomes hydrophilic whereby the thermo-responsive polymer is contracted due to the dehydration.

When the alternate control for switching the first flow 24 and second flow 25 is performed, the flow having different temperature is entered into the flow channel 22 so as to change the temperature of the porous thermo-responsive layer 20 periodically. Once the temperature of the porous thermo-responsive layer 20 is varied above or below the LCST, the porous thermo-responsive layer 20 can generate an expansion movement or contraction movement thereby simulating the organ operation of live being. The bionic device provided in the present invention can be utilized to replace the convention animal experiments and reducing the cost of drug test and clinical trial. It is noted the first flow and the second flow can be the same liquid or gas, or can be liquid or gas different from each other.

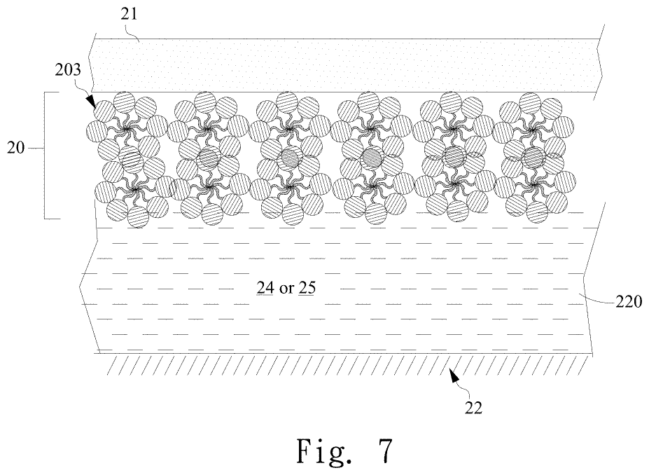

Please refer to FIG. 8, which illustrates a method for forming the bionic device according one embodiment of manufacturing flow of the present invention. In the present embodiment, taking structure shown in FIGS. 2A and 2B, the step is started by step 50 for forming a porous thermo-responsive layer 20 having a plurality of thermo-responsive polymer molecules. In the step 50, the porous thermo-responsive layer 20 has a first surface 200, and a second surface 201 opposite to the first surface 200. In one embodiment for forming the porous thermo-responsive layer 20, an electro-spinning method is utilized for making spinning from the thermo-responsive polymer molecules, in which a liquid having the thermo-responsive polymer molecules, alcohol, optical cured agent, and optical cross-link agent is made for electro-spinning procedure. The detail of electro-spinning is well known by the one having ordinary skilled in the art, and it will not be described hereinafter. The diameter of the spinning can be, but should not be limited to, 1.5 um. After that, the spinning is weaved to form the thermo-responsive layer 20 on a substrate. Please refer to FIGS. 3A to 3C, the thermo-responsive layer can be formed by vertically weaved procedure, horizontally weaved procedure, and randomly weaved procedure. After weaving procedure, the thermo-responsive layer 20 can be removed from the substrate.

Next, in the step 51, a cell culturing layer 21 is formed on the porous thermo-responsive layer 20. The combination of porous thermo-responsive layer 20 and the cell culturing layer 21 is regarded as a bionic structure. In one embodiment of step 51, a PEI material is coated onto the porous thermo-responsive layer 20 for forming the cell culturing layer 21. Alternatively, like the embodiment shown in FIG. 3E, the substrate 206 having the porous thermo-responsive layer 20 is directly soaked into the PEI liquid for forming the cell culturing layer onto porous the thermo-responsive layer, and then the combination of porous thermo-responsive layer and the cell culturing layer is removed from the substrate. Alternatively, the PEI liquid is coated onto the thermo-responsive layer formed on the substrate in the step 50, and then, the combination of porous thermo-responsive layer and the cell culturing layer is removed from the substrate.

When the step 51 is finished, a step 52 is performed to arrange the bionic structure into the accommodating space 220 in the flow channel 22, whereby at least one flow 24 or 25 is capable of directly or indirectly passing through the second surface of the porous thermo-responsive layer. Finally, a step 53 is performed to provide a control module 23, such as the modules shown in FIG. 2A, 2D or 2E, for example. The control module 23 is coupled to the flow channel 22. In the embodiment, shown in FIG. 2A, the control module 23 is utilized to switch the flows having different temperature passing through the second surface of the porous thermo-responsive layer whereby a temperature of the porous thermo-responsive layer is varied above or below a critical temperature with respect to an expansion movement or contraction movement of the thermo-responsive material. Through the alternately switching the flows having different temperature, the porous thermo-responsive layer can perform expansion and contraction movement periodically. Alternatively, in the FIGS. 2D and 2E, the control module 23a or 23b is utilized to control the temperature of the liquid sealed inside the space 220 corresponding to the porous thermo-responsive layer.

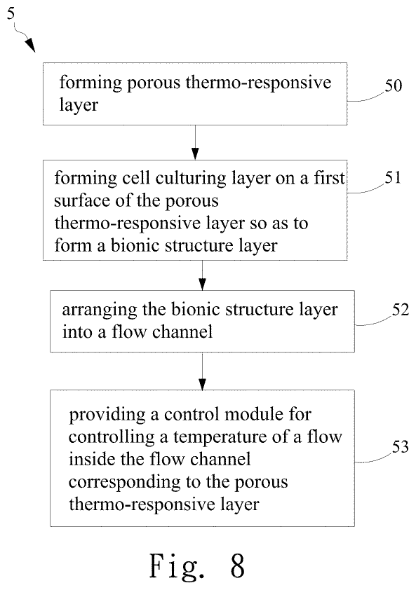

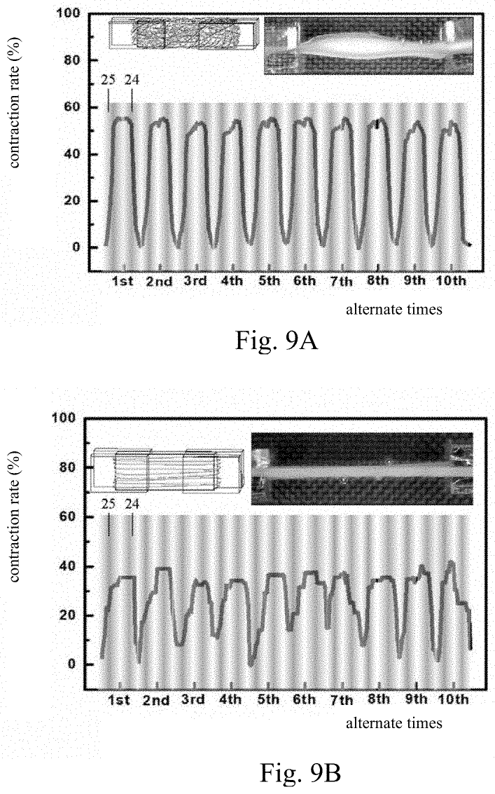

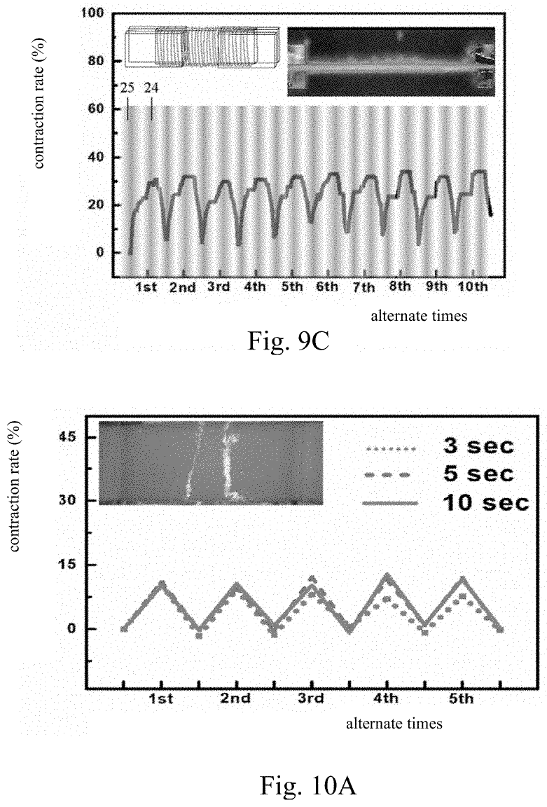

Please refer to FIGS. 9A to 9C, which illustrate a curve with respect to contracting rate of the porous thermo-responsive layer having different weaving measures. It is noted that a procedure for calculating contracting rate is to monitor and record thickness variation of the sample through charge-coupled device (CCD) a plurality of times “N”. In the present embodiment, the N is 10. Then a contracting rate can be calculated through the equation shown below, wherein Vₙ represents a thickness value at N^{th} time and Vₘₐₓ represents the maximum thickness value among the 10 tests.

$$Swelling Ratio = \frac{{V}_{n} - {V}_{max}}{{V}_{max}} \times 100 \%$$

In the FIGS. 9A to 9C, the vertical axis represents the contraction rate along the thickness direction while the horizontal axis represents the period for alternately switching the first and second flow. In the FIG. 9A, it shows a curve of contraction rate of randomly weaved porous thermo-responsive layer. In the FIG. 9B, it shows a curve of contraction rate of vertically weaved porous thermo-responsive layer. In the FIG. 9C, it shows a curve of contraction rate of horizontally weaved porous thermo-responsive layer. According to the result shown in FIGS. 9A to 9C, the contraction rate of the randomly weaved porous thermo-responsive layer is more stable and better than the contraction rate of the vertically weaved or horizontally weaved porous thermo-responsive layer.

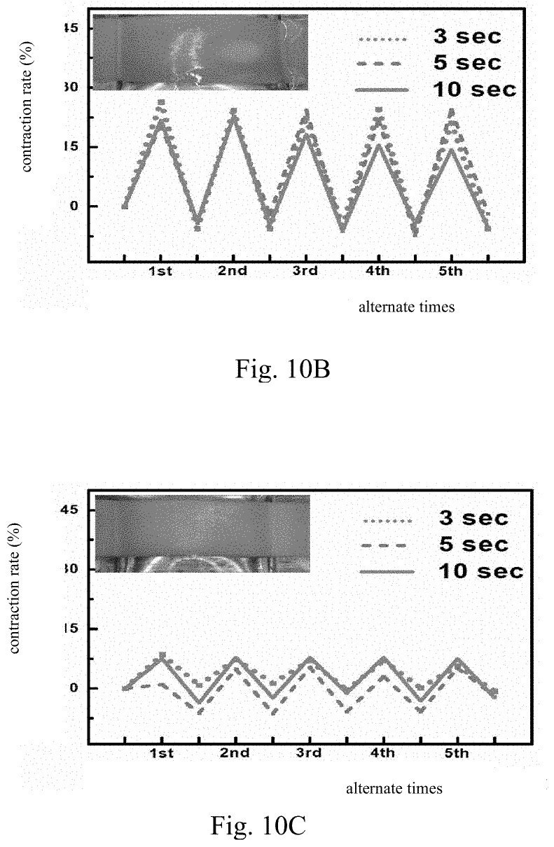

Please refer to FIGS. 10A to 10C, which represent a variation of contraction rate during the expansion and contraction movement when first and second flow are switched to passing through the porous thermo-responsive layer having different weaving measures, respectively. According to the result shown in FIGS. 10A to 10 C, it is clear that when the smaller thickness of the porous thermo-responsive layer is, the higher contraction rate could be generated. In addition, the contraction rate of the randomly weaved porous thermo-responsive layer is better than the horizontally or vertically weaved porous thermo-responsive layer. In addition, it is also known that the time interval with ten seconds for alternate expansion and contraction movement has better contraction rate than 3 or five seconds. The experimental result shown in FIGS. 9A to 10C is only utilized to explain the concept of the present invention is practical, and it will not be utilized to limit the scope of the present invention.

While the present invention has been particularly shown and described with reference to a preferred embodiment, it will be understood by those skilled in the art that various changes in form and detail may be without departing from the spirit and scope of the present invention.

What is claimed is:

1. A bionic organic device, comprising:
a porous thermo-responsive layer, configured to have a porous structure formed by a plurality of thermo-responsive polymer molecules, the porous thermo-responsive layer comprising a first surface and a second surface opposite to the first surface;
a cell culturing layer, formed on the first surface of the porous thermo-responsive layer;
a flow channel, configured to have an accommodating space for accommodating the porous thermo-responsive layer, and at least one flow contacting with the second surface of the porous thermo-responsive layer; and
a control module, configured to control a temperature of the flow for affecting the porous thermo-responsive layer so that the temperature of the porous thermo-responsive layer is changed to be above or below a critical temperature of the porous thermo-responsive layer by the control module whereby an expansion movement and contraction movement is generated by the porous thermo-responsive layer.

2. The device of claim 1, wherein the control module is a flow control module for switching flows having different temperature passing through a space corresponding to the second surface of the porous thermo-responsive layer.

3. The device of claim 2, wherein the flow control module further comprises:
a first flow, configured to have a first temperature less than or equal to an expansion temperature or a contraction temperature of the thermo-responsive polymer molecules;
a second flow, configured to have a second temperature greater than or equal to the expansion temperature or the contraction temperature of the thermo-responsive polymer molecules; and
a valve module, coupled to the flow channel for selectively communicating the first flow or the second flow with the flow channel thereby changing the temperature of the porous thermo-responsive layer.

4. The device of claim 1, wherein the porous structure is formed by weaving a spinning formed by the plurality of thermo-responsive polymer molecules, wherein a way of weaving the spinning is vertical weaving, horizontal weaving or random weaving.

5. The device of claim 1, wherein the porous thermo-responsive layer further comprises a porous substrate having the plurality of thermo-responsive polymer molecules formed thereon.

6. The device of claim 1, wherein a cell culturing fluid is arranged on the top of the cell culturing layer formed in the flow channel whereby the cell culturing layer absorbs a plurality of cells from the cell culturing fluid.

7. The device of claim 1, wherein the critical temperature is lower critical solution temperature (LCST) or upper critical solution temperature (UCST).

8. The device of claim 1, wherein a housing is formed to defined the flow channel so that the flow is enclosed within the flow channel, and the control module is an energy controlling module for controlling the temperature of the flow.

9. A method for forming a bionic organic device, comprising steps of:
forming a porous thermo-responsive layer having a plurality of thermo-responsive polymer molecules, the porous thermo-responsive layer comprising a first surface and a second surface opposite to the first surface;
forming a cell culturing layer on the first surface of the porous thermo-responsive layer so that the cell culturing layer and the porous thermo-responsive layer are formed as a bionic structure layer;
arranging the bionic structure layer into a flow channel having an accommodating space for accommodating the porous thermo-responsive layer, wherein the flow channel is configured to accommodating at least one flow corresponding to the second surface of the porous thermo-responsive layer; and
controlling a temperature of the flow for affecting the porous thermo-responsive layer by using a control module coupled to the flow channel whereby a temperature of the porous thermo-responsive layer is changed to be above or below a critical temperature of the porous thermo-responsive layer so as to generate a contraction or an expansion movement.

10. The method of claim 9, wherein the control module is a flow control module utilized to switch flows respectively having different temperature passing through a space corresponding to the second surface of the porous thermo-responsive layer.

11. The method of claim 9, wherein the flow control module further comprises:
a first flow, configured to have a first temperature less than or equal to the critical temperature with respect to the expansion movement and contraction movement of the porous thermo-responsive layer;
a second flow, configured to have a second temperature greater than the first temperature, wherein the second temperature is greater than or equal to the critical temperature with respect to expansion movement and contraction movement of the porous thermo-responsive layer; and
a valve module, configured to couple to the flow channel for selecting the first flow or the second flow to enter the flow channel thereby changing the temperature of the porous thermo-responsive layer.

12. The method of the claim 9, wherein the step for forming the porous thermo-responsive layer further comprises steps of:
forming a spinning by using the thermo-responsive polymer molecules; and
weaving the spinning to form the porous thermo-responsive layer, wherein the spinning is vertically weaved, horizontally weaved or randomly weaved.

13. The method of claim 9, wherein a housing is formed to defined the flow channel so that the flow is enclosed within the flow channel, and the control module is an energy controlling module for controlling the temperature of the flow.

14. The method of claim 9, wherein the step for forming the porous thermo-responsive layer is a self-assembly thermo-responsive layer formed by stacking the plurality of thermo-responsive polymer molecules.

15. The method of claim 14, wherein the step of forming the self-assembly thermo-responsive layer further comprises steps of:
making the temperature of the porous thermo-responsive layer greater than the critical temperature with respect to expansion movement and contraction movement of the porous thermo-responsive layer, whereby the thermo-responsive polymer molecules are contracted to form spaces between the thermo-responsive polymer molecules; and
forming an adhesive layer on the contracted thermo-responsive polymer molecules.

16. The method of claim 15, wherein the adhesive layer is ultraviolet adhesive cured by using the ultraviolet rays.

17. The method of claim 9, wherein the step for forming the porous thermo-responsive layer further comprises steps of:
providing a porous substrate; and
forming a thermo-responsive layer onto the substrate by titrating or spinning coating process.

18. The method of claim 17, further comprising a step of forming the porous thermo-responsive layer by using a mold pressing onto the thermo-responsive layer.

19. The method of claim 9, wherein the critical temperature is lower critical solution temperature (LCST) or upper critical solution temperature (UCST).

20. The method of claim 9, wherein a cell culturing fluid is arranged on the top of the cell culturing layer in the flow channel whereby the cell culturing layer absorbs a plurality of cells from the cell culturing fluid.
